(19) United States
(12) Patent Application Publication
(10) Pub. No.: US 2026/0028447 A1
(43) Pub. Date: Jan. 29, 2026

(54) POLYMER, THIOPHENE COMPOUND, CONDUCTIVE MEMBER, AND ELECTRONIC PART

(71) Applicant: TDK CORPORATION, Tokyo (JP)

(72) Inventor: Tatsuya ABE, Tokyo (JP)

(73) Assignee: TDK CORPORATION, Tokyo (JP)

(21) Appl. No.: 18/998,884

(22) PCT Filed: Jul. 27, 2023
(86) PCT No.: PCT/JP2023/027499

§ 371 Date: Jan. 27, 2025

(30) Foreign Application Priority Data
Jul. 29, 2022 (JP) 2022-121673
Feb. 16, 2023 (JP) 2023-022716

(51) Int. Cl. C08G 61/12; C07D 495/04

(52) U.S. Cl. CPC C08G 61/126; C07D 495/04; C08G 2261/1424; C08G 2261/3243; C08G 2261/43; C08G 2261/794

(57) ABSTRACT

A polymer that includes at least one structural unit selected from structural units represented by Formula (1) and Formula (2) (in the formulae, X is a divalent linking group. A is a halogen atom or an alkyl group having 1 to 5 carbon atoms which may have a substituent, and M is a cation).

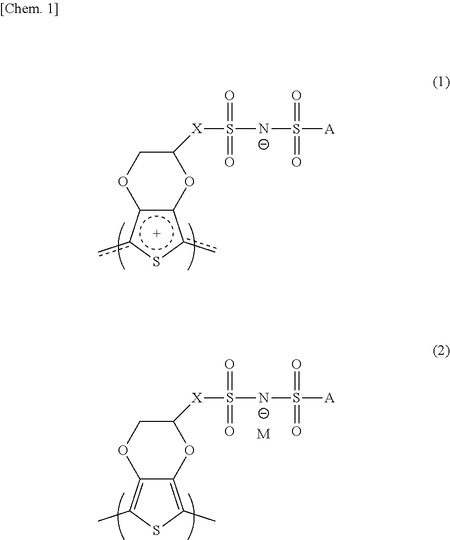

### TECHNICAL FIELD

The present invention relates to a polymer, a thiophene compound, a conductive member, and an electronic part.

Priority is claimed on Japanese Patent Application No. 2022-121673, filed Jul. 29, 2022, and Japanese Patent Application No. 2023-022716, filed Feb. 16, 2023, the contents of which are incorporated herein by reference.

### BACKGROUND ART

A conductive polymer has conductivity and also has organic properties (light weight, flexibility, humidity sensing ability, and the like) that differ from metals. For this reason, the conductive polymer is being researched for application to electronic parts that are used in a wide range of fields, such as a sensor, a capacitor, an actuator, a lithium ion secondary battery, and a bioelectrode.

The conductive polymer is a compound having a π-electron conjugated structure. In general, a conductive polymer alone has poor conductivity. For this reason, a conductive polymer is doped with a compound (dopant) having high acceptor properties or donor properties to exhibit conductivity. In a case where a conductive polymer is doped with a dopant, that serves as an acceptor, a π electron is extracted from the π electron conjugated structure to generate a positive hole or a hole, thereby conductivity being exhibited.

In addition, in a case where a conductive polymer is doped with a dopant that serves as a donor, an electron is supplied from the donor to the π-electron conjugated structure to generate a negative charge carrier having a negative charge, thereby conductivity being exhibited.

In recent years, attention has been focused on a self-doped type conductive polymer as a conductive polymer, which exhibits conductivity without being doped with a dopant from the outside. The self-doped type conductive polymer has a chain structure having a π-electron conjugated structure, to which a group having a dopant function is bonded. The self-doped type conductive polymer is not required to be doped with a dopant from outside, and dopant migration does not occur. For this reason, the self-doped type conductive polymer is excellent in stability as compared with a conductive polymer doped with a dopant.

Examples of the self-doped type conductive polymer include those described in Patent Document 1 and Patent Document 2.

Patent Document 1 describes a thiophene compound in which a group having, at the terminal, —SO₃M (M represents a hydrogen atom, an alkali metal selected from the group consisting of Li, Na, and K, NH(R¹)₃, or HNC₅H₅), where R¹'s each independently represent a hydrogen atom or an alkyl group having 1 to 6 carbon atoms which may have a substituent) is bonded to a carbon atom in an ethylenedioxy group included in a structural unit containing 3,4-ethylenedioxythiophene, polythiophenes obtained by polymerizing this thiophene compound, and aqueous solutions of these polythiophenes.

Patent Document 2 describes, as a structural unit of a polythiophene, a structural unit in which a substituent is bonded to a carbon atom in an ethylenedioxy group included in a structural unit containing 3,4-ethylenedioxythiophene. The substituents described are a group having, at the terminal, —C(R)SO₃M (R represents a chain-like or branched alkyl group having 1 to 6 carbon atoms, or a fluorine atom, M represents a hydrogen atom, an alkali metal selected from the group consisting of Li, Na, and K, NH(R¹)₃, or HNC₅H₅), where R¹'s each independently represent a hydrogen atom or an alkyl group having 1 to 6 carbon atoms which may have a substituent), and a group having, at the terminal, —C(R)SO₃⁻ (R represents a chain-like or branched alkyl group having 1 to 6 carbon atoms, or a fluorine atom). In addition, Patent Document 2 describes an aqueous solution of the polythiophene.

### CITATION LIST

### Patent Documents

[Patent Document 1] Japanese Patent No. 6040615

[Patent Document 2] Japanese Patent No. 6131780

### SUMMARY OF INVENTION

### Technical Problem

However, the self-doped type conductive polymer in the related art is water-soluble. For this reason, in a case where a conductive member containing the self-doped type conductive polymer in the related art comes into contact with water, the self-doped type conductive polymer elutes, which may make it not possible to maintain the shape or make conductivity decrease. For this reason, there has been a demand for improving water resistance in a conductive member containing the self-doped type conductive polymer in the related art and in an electronic part using such a conductive member.

The present invention has been made in consideration of the above circumstances, and an object of the present invention is to provide a self-doped type conductive polymer that has low hydrophilicity and good lipophilicity, where the self-doped type conductive polymer is a polymer that is capable of forming a conductive member having sufficient conductivity and excellent water resistance.

In addition, another object of the present invention is to provide a thiophene compound that can be used as a raw material monomer of a self-doped type conductive polymer that has sufficient conductivity, has low hydrophilicity, and has good lipophilicity.

In addition, still another object of the present invention is to provide a conductive member that contains the polymer according to the present invention and has excellent water resistance, and an electronic part that contains this conductive member.

### Solution to Problem

In order to solve the above problems, the following means are provided.

A polymer according to one aspect of the present invention is a polymer containing at least one structural unit selected from a structural unit represented by General Formula (1) and a structural unit represented by General Formula (2).

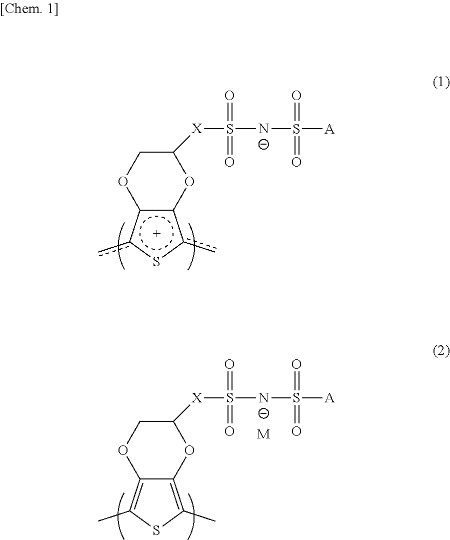

(In General Formula (1), X represents a divalent linking group, A represents a halogen atom or an alkyl group having 1 to 5 carbon atoms which may have a substituent.)

(In General Formula (2), X represents a divalent linking group, A represents a halogen atom or an alkyl group having 1 to 5 carbon atoms which may have a substituent, and M represents a cation.)

### Advantageous Effects of Invention

The polymer according to the present invention contains at least one structural unit selected from a structural unit represented by General Formula (1) and a structural unit represented by General Formula (2). For this reason, the polymer according to the present invention is a self-doped type conductive polymer having low hydrophilicity and good lipophilicity, and it can be suitably used as a material that is capable of forming a conductive member having sufficient conductivity and excellent water resistance.

In addition, the thiophene compound according to the present invention is represented by General Formula (3). For this reason, in a case of polymerizing a raw material monomer containing the thiophene compound according to the present invention, it is possible to produce the polymer according to the present invention, which is a self-doped type conductive polymer that has sufficient conductivity, has low hydrophilicity, and has good lipophilicity.

In addition, the conductive member according to the present invention and an electronic part including the conductive member contains the polymer according to the present invention. For this reason, the conductive member according to the present invention and electronic part including the conductive member have excellent water resistance.

### BRIEF DESCRIPTION OF DRAWINGS

FIG. 1 is a graph showing the results obtained by analyzing a polymer of Example 1 by an attenuated total reflection method using a Fourier transform infrared spectrophotometer (FT-IR).

FIG. 2 is a ¹H-NMR measurement chart of a thiophene compound synthesized in Example 1.

FIG. 3 is a graph showing the results obtained by analyzing the polymer of Example 1 by gel permeation chromatography (GPC).

### DESCRIPTION OF EMBODIMENTS

In order to solve the above problems, the inventors of the present invention focused on a substituent having a dopant function, which is bonded to a carbon atom in an ethylenedioxy group included in a structural unit containing 3,4-ethylenedioxythiophene, in a self-doped type conductive polymer having a structural unit containing 3,4-ethylenedioxythiophene, and carried out extensive studies.

As a result, the inventors of the present invention found that it is sufficient to obtain a polymer containing a structural unit having a group in which as a substituent having a dopant function, a specific imide group is bonded, through a divalent linking group, to a carbon atom in the ethylenedioxy group of 3,4-ethylenedioxythiophene.

This polymer is allowable to be a self-doped type conductive polymer having excellent water resistance because the substituent having a dopant function, which is bonded to a carbon atom in an ethylenedioxy group included in a structural unit containing 3,4-ethylenedioxythiophene, is a group in which a specific imide group is bonded through a divalent linking group. As a result, it is presumed that in a conductive member containing a polymer having the above-described substituent having a dopant function, excellent water resistance is obtained as compared with, for example, a conductive member containing a polymer having a group containing a sulfonate group such as —SO₃Na instead of an imide group contained in the above-described substituent having a dopant function.

Further, the inventors of the present invention manufactured a conductive member that contains a polymer containing a structural unit having a group in which a specific imide group is bonded, through a divalent linking group, to a carbon atom in the ethylenedioxy group of 3,4-ethylenedioxythiophene. Then, the inventors confirmed that the conductive member has good water resistance and sufficient conductivity and arrived at the present invention.

The present invention includes the following aspects.

[1] A polymer containing:
at least one structural unit selected from a structural unit represented by General Formula (1) and a structural unit represented by General Formula (2).

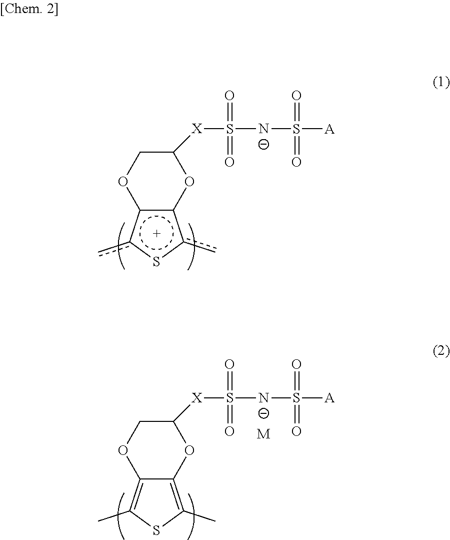

(In General Formula (1), X represents a divalent linking group, A represents a halogen atom or an alkyl group having 1 to 5 carbon atoms which may have a substituent.)

(In General Formula (2), X represents a divalent linking group, A represents a halogen atom or an alkyl group having 1 to 5 carbon atoms which may have a substituent, and M represents a cation.)

[2] The polymer according to [1],
wherein in the structural unit represented by General Formula (1) and the structural unit represented by General Formula (2), X is a divalent linking group represented by General Formula (L—Ar),
(in General Formula (L—Ar), L represents a single bond or a divalent linking group, and Ar represents any one selected from a phenylene group which may have a substituent, a biphenylene group which may have a substituent, or a naphthylene group which may have a substituent).

[3] The polymer according to [2],
wherein L is represented by General Formula (L-a),
Ar is a phenylene group, and
A is a fluorine atom or an alkyl group having 1 to 5 carbon atoms, in which at least a part of hydrogen atoms are substituted with a fluorine atom.

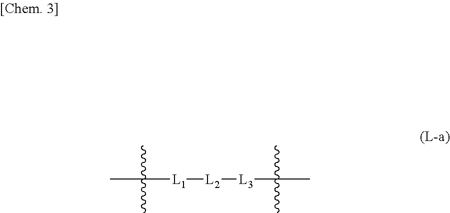

(In General Formula (L-a), L₁, L₂, and L₃ each independently represent any one selected from a single bond, an alkylene group having 1 to 2 carbon atoms, or an ether bond.)

[4] The polymer according to [1],
wherein in the structural unit represented by General Formula (1) and the structural unit represented by General Formula (2), X has a chain structure which is obtained by subjecting a plurality of alkylene groups to ether bonding.

[5] A thiophene compound represented by General Formula (3).

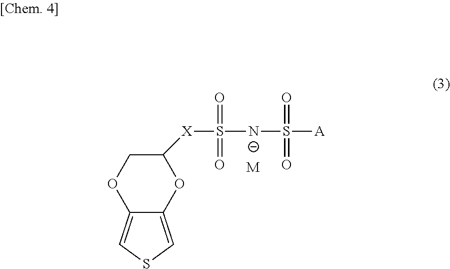

(In General Formula (3), X represents a divalent linking group, A represents a halogen atom or an alkyl group having 1 to 5 carbon atoms which may have a substituent, and M represents a cation.)

[6] The thiophene compound according to [5],
wherein, in General Formula (3), X is a divalent linking group represented by General Formula (L—Ar),
(in General Formula (L—Ar), L represents a single bond or a divalent linking group, and Ar represents any one selected from a phenylene group which may have a substituent, a biphenylene group which may have a substituent, or a naphthylene group which may have a substituent).

[7] The thiophene compound according to [6],
wherein, L is represented by General Formula (L-a),
Ar is a phenylene group, and
A is a fluorine atom or an alkyl group having 1 to 5 carbon atoms, in which at least a part of hydrogen atoms are substituted with a fluorine atom.

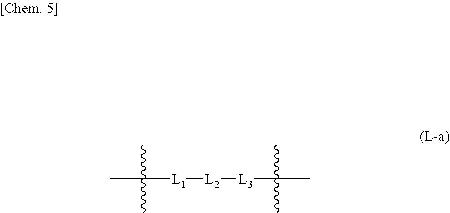

(In General Formula (L-a), L₁, L₂, and L₃ each independently represent any one selected from a single bond, an alkylene group having 1 to 2 carbon atoms, or an ether bond.)

[8] The thiophene compound according to [5],
wherein in General Formula (3), X has a chain structure which is obtained by subjecting a plurality of alkylene groups to ether bonding.

[9] A conductive member containing:
the polymer according to any one of [1] to [4].

[10] An electronic part including:
the conductive member according to [9].

Hereinafter, the polymer, the thiophene compound, the conductive member, and the electronic part according to the present invention will be described in detail.

### Polymer

The polymer (self-doped type conductive polymer) according to the present embodiment contains at least one structural unit selected from a structural unit represented by General Formula (1) and a structural unit represented by General Formula (2). The polymer according to the present embodiment is such that in a solid state, a state of the structural unit represented by General Formula (1) and a state of the structural unit represented by General Formula (2) are in equilibrium. The ratio of the structural unit represented by General Formula (1) to the structural unit represented by General Formula (2) in the polymer according to the present embodiment is not particularly limited.

The structural unit represented by General Formula (1) indicates a doped state of the structural unit represented by General Formula (2). In the polymer according to the present embodiment, an imide salt (—SO₂—N⁻M—SO₂A) contained in the substituent (—X—SO₂—N⁻M—SO₂A) having a dopant function in the structural unit represented by General Formula (2) serves as a dopant. The cation (M) in the structural unit represented by General Formula (2), which is contained in the polymer according to the present embodiment, may be present in the polymer in a state of a cation or may be present in a state of being bonded to the nitrogen atom in the imide salt.

In the structural unit represented by General Formula (1), the imide salt contained in the structural unit represented by General Formula (2) is doped to deprive a π electron from the π electron conjugated structure of the thiophene ring that forms the main chain of the polymer, whereby a positive charge (a positive hole or a hole) is generated. That is, the polymer according to the present embodiment is a self-doped type conductive polymer that is capable of obtaining conductivity without being doped with a dopant from the outside.

In the polymer according to the present embodiment, the substituent (—X—SO₂—N⁻—SO₂A) having a dopant function in the structural unit represented by General Formula (1) and the substituent (—X—SO₂—N⁻M—SO₂A) having a dopant function in the structural unit represented by General Formula (2) may be bonded to any of the two carbon atoms of the ethylenedioxy group of 3,4-ethylenedioxythiophene. In addition, in a plurality of structural units that form the polymer according to the present embodiment, all of the bonding positions of the substituents having a dopant function to the carbon atoms contained in the ethylenedioxy group may be the same, or a part thereof may be different from each other.

In the structural unit represented by General Formula (1) and the structural unit represented by General Formula (2) which are contained in the polymer according to the present embodiment, X represents a divalent linking group. It is preferable that X is a chain structure containing any one or more groups selected from an arylene group, an alkylene group, and an ether bond in any order, where the number of the groups is one or plural. It is preferable that X contains an arylene group in order to be allowable to be a self-doped type conductive polymer that has still lower hydrophilicity and still better lipophilicity. In a case where X contains an arylene group, it is more preferable that the arylene group contained in X is bonded to the sulfur atom of the imide salt.

In the structural unit represented by General Formula (1) and the structural unit represented by General Formula (2), it is preferable that X is a divalent linking group represented by General Formula (L—Ar). In General Formula (L—Ar), L represents a single bond or a divalent linking group, and Ar represents any one selected from a phenylene group which may have a substituent, a biphenylene group which may have a substituent, or a naphthylene group which may have a substituent. In General Formula (L—Ar), the bonding site on the left side is bonded to a carbon atom in an ethylenedioxy group included in a structural unit containing 3,4-ethylenedioxythiophene, and the bonding site on the right side is bonded to the sulfur atom of the imide salt in the polymer according to the present embodiment.

In a case where L in the divalent linking group represented by General Formula (L—Ar) is a divalent linking group, the self-doped type conductive polymer is allowable to have good water resistance as compared with in a case where L is a single bond, which is preferable. In a case where Lis a divalent linking group, it is preferable that L is a divalent linking group having a chain structure consisting of one or plurality of alkylene groups and one or plurality of ether bonds, where it is more preferable the divalent linking group has 1 to 8 carbon atoms. In a case where L is a divalent linking group, it is preferable that the divalent linking group has 8 or less carbon atoms since it is not allowable for the chain structure of the divalent linking group to be too long, which makes it difficult for the molecular chains to be arranged in the polymer according to the present embodiment and prevents the conductivity of the polymer from becoming insufficient. Examples of divalent linking groups having 8 or less carbon atoms include —(CH₂)₁₋₈O—, —O(CH₂)₁₋₈—, —O(CH₂)₁₋₈O—, —O(CH₂)₂O(CH₂)₂—, —CH₂O(CH₂)₂OCH₂—, —CH₂O(CH₂)₂O—, and —CH₂OCH₂—.

In the divalent linking group represented by General Formula (L—Ar), L is preferably a divalent linking group represented by General Formula (L-a). In General Formula (L-a), L₁ is bonded to a carbon atom of an ethylenedioxy group in a structural unit containing 3,4-ethylenedioxythiophene, and L₃ is bonded to Ar. In General Formula (L-a), L₁, L₂, and L₃ are each independently any one selected from a single bond, an alkylene group having ₁ to ₂ carbon atoms, or an ether bond. Specific examples thereof include —CH₂OCH₂— (L₁=an alkylene group having 1 carbon atom, L₂=an ether bond, and L₃=an alkylene group having 1 carbon atom), —CH₂O — (L₁=an alkylene group having 1 carbon atom, L₂=an ether bond, and L₃=a single bond), —(CH₂)₂O— (L₁=an alkylene group having 2 carbon atoms, L₂=an ether bond, and L₃=a single bond), —O(CH₂)₁₋₂— (L₁=an ether bond, L₂=an alkylene group having 1 to 2 carbon atoms, and L₃=a single bond), —O(CH₂)₁₋₂—O— (L₁=an ether bond, L₂=an alkylene group having 1 to 2 carbon atoms, and L₃=an ether bond), and —CH₂OCH₂— or —CH₂O— is preferable. This is because the polymer can be easily produced.

In the structural unit represented by General Formula (1) and the structural unit represented by General Formula (2) which are contained in the polymer according to the present embodiment, Ar represents any one selected from a phenylene group which may have a substituent, a biphenylene group which may have a substituent, and a naphthylene group which may have a substituent. Ar is preferably an unsubstituted phenylene group because it can be easily produced.

Examples of the substituents in the phenylene group which may have a substituent, the biphenylene group which may have a substituent, and the naphthylene group which may have a substituent include a fluorine atom, a methyl group, an ethyl group, a methoxy group, an ethoxy group, an acetyl group, a methyl ester group, and a cyano group.

In the structural unit represented by General Formula (1) and the structural unit represented by General Formula (2) which are contained in the polymer according to the present embodiment, X may have a chain structure which is obtained by subjecting a plurality of alkylene groups to ether bonding. In this case, the total number of carbon atoms and oxygen atoms contained in the chain structure is preferably 3 to 20. This is because in a case where the total number of carbon atoms and oxygen atoms is 20 or less, it is not allowable for the chain structure to be too long, which makes it difficult for the molecular chains to be arranged in the polymer according to the present embodiment and prevents the conductivity of the polymer from becoming insufficient. The total number of carbon atoms and oxygen atoms contained in the chain structure is more preferably 3 to 12. Specific examples of such a chain structure include —CH₂O(CH₂)₂OCH₂—, —CH₂O(CH₂)₃OCH₂—, —CH₂O(CH₂)₃—, —CH₂OCH₂—, —(CH₂)₂OCH₂—, and —CH₂O(CH₂)₂—, —CH₂O(CH₂)₄—, and among these, —CH₂O(CH₂)₂—, —CH₂O(CH₂)₃—, or —CH₂O(CH₂)₄— is preferable since the synthesis of the polymer can be easily carried out. In these specific chain structures, the bonding site on the left side is bonded to a carbon atom in an ethylenedioxy group included in a structural unit containing 3,4-ethylenedioxythiophene, and the bonding site on the right side is bonded to the sulfur atom of the imide salt in the polymer according to the present embodiment.

In the structural unit represented by General Formula (1) and the structural unit represented by General Formula (2) which are contained in the polymer according to the present embodiment, A represents a halogen atom or an alkyl group having 1 to 5 carbon atoms which may have a substituent.

Examples of the substituent in the alkyl group having 1 to 5 carbon atoms which may have a substituent include a halogeno group such as a fluoro group, a chloro group, or a bromo group, an alkyl group such as a methyl group or an ethyl group, an alkoxy group such as a methoxy group or an ethoxy group, and a cyano group.

In the polymer according to the present embodiment, A is preferably a fluorine atom or an alkyl group having 1 to 5 carbon atoms, in which at least a part of hydrogen atoms are substituted with a fluorine atom, and it is more preferably an alkyl group having 1 to 3 carbon atoms, in which all of the hydrogen atoms are substituted with a fluorine atom (perfluoroalkyl group). This is because the self-doped type conductive polymer is allowable to have still lower hydrophilicity and still better lipophilicity, and thus the polymer is capable of forming a conductive member having still better water resistance.

In the structural unit represented by General Formula (2) according to the present embodiment, M represents a cation, and examples thereof include —Li⁺, —Na⁺, —K⁺, —H⁺, pyridinium (—[C₅H₅HN]⁺), and an ammonium ion (—N⁺H(R¹)₃ (three R¹'s in the formula each independently represent a hydrogen atom or an alkyl group having 1 to 15 carbon atoms which may have a substituent)). Specific examples of the ammonium ion which is the cation (M) include —N⁺H₃C₂H₄OH (two of the three R¹'s in the above formula —N⁺H(R¹)₃ are a hydrogen atom, where the remaining one R¹ is an ethyl group having one hydroxyl group as a substituent). In order for the self-doped type conductive polymer to be allowable to have better conductivity, M is preferably any one selected from —Na⁺, —H⁺, —N⁺H₃C₂H₄OH and is particularly preferably —H⁺.

In the polymer according to the present embodiment, the substituent (—X—SO₂—N⁻M—SO₂A) that has a dopant function and is bonded to a carbon atom, in the ethylenedioxy group of 3,4-ethylenedioxythiophene, is a group to which an imide salt (—SO₂—N⁻M—SO₂A) is bonded through a divalent linking group (X). For this reason, even in a case where the cation (M) contained in the substituent having a dopant function is a cation such as —Na⁺ or —K⁺, which is likely to easily provide a highly hydrophilic polymer, the self-doped type conductive polymer is allowable to have excellent water resistance.

The degree of polymerization of the polymer according to the present embodiment is not particularly limited; however, it is preferably in a range of 3 to 5,000. In a case where the degree of polymerization of the polymer is 3 or more, the self-doped type conductive polymer is allowable to have more excellent water resistance. The degree of polymerization of the polymer is more preferably 10 or more. In a case where the degree of polymerization of the polymer is 10 or more, the self-doped type conductive polymer is allowable to have still better water resistance and better conductivity. In addition, in a case where the degree of polymerization of the polymer is 5,000 or less, the self-doped type conductive polymer is allowable to have sufficient solubility in a solvent. For this reason, the adaptability and productivity of the process are excellent in a case of producing a conductive member containing the polymer according to the present embodiment.

### “Method of Producing Polymer”

### “Thiophene Compound”

To produce the polymer according to the present embodiment, first, a raw material monomer containing a thiophene compound represented by General Formula (3) is prepared.

X, A, and M in the thiophene compound represented by General Formula (3) are respectively the same as X, A, and M in the structural unit represented by General Formula (2).

### “Method of Producing Thiophene Compound”

The thiophene compound represented by General Formula (3) can be produced, for example, by the method shown below. First, a halogen compound (D₁—X₀—SO₂—D₂ (D₁ and D₂ are halogens, and X0 is a part of X in General Formula (2)) having a structure corresponding to a part of the substituent having a dopant function in the structural unit represented by General Formula (2), is prepared by a method publicly known in the related art.

It is possible to use as the halogen compound, for example, 4-(bromomethyl)benzenesulfonyl chloride, 4-(chloromethyl)benzenesulfonyl chloride, 4-fluorobenzenesulfonyl chloride, 4-(2-bromoethoxy)benzenesulfonyl chloride, and 3-chloropropanesulfonyl chloride.

The halogen compound may be synthesized by a publicly known method, or a commercially available halogen compound may be used.

Next, D₂ in the above halogen compound is reacted with NH₂ in the sulfonamide (NH₂—SO₂—A (A is the same as A in General Formula (2))) having a terminal group corresponding to A in General Formula (2) in the presence of a compound containing a cationic species that serves as M in General Formula (2) to obtain an intermediate compound.

It is possible to use as the sulfonamide having a terminal group corresponding to A, for example, trifluoromethanesulfonamide, pentafluoroethanesulfonamide, heptafluoropropanesulfonamide, 1, 1,2,2,3,3,4,4,4-nonafluorobutanesulfonamide, methanesulfonamide, and ethanesulfonamide.

The sulfonamide having a terminal group corresponding to the above A may be synthesized by a publicly known method, or a commercially available product may be used.

In a case where M in General Formula (2) is Na⁺, it is possible to use as the compound containing a cation species that serves as M, for example, anhydrous sodium carbonate and sodium hydroxide.

In a case where M in General Formula (2) is K⁺, it is possible to use as the compound containing a cation species that serves as M, for example, anhydrous potassium carbonate and potassium hydroxide.

Next, the obtained intermediate compound is reacted with a compound in which a group corresponding to a part of X in General Formula (2) is bonded to a carbon atom in the ethylenedioxy group of 3,4-ethylenedioxythiophene. As a result, a thiophene compound represented by General Formula (3), in which the substituent (—X—SO₂—N⁻M—SO₂A) having a dopant function is bonded to a carbon atom in the ethylenedioxy group of 3,4-ethylenedioxythiophene, is obtained.

It is possible to use, for example, (2,3-dihydrothieno[3,4-b][1,4]dioxin-2-yl)methanol as the compound in which a group (for example, a hydroxyl group) corresponding to a part of X in General Formula (2) is bonded to the carbon atom in the ethylenedioxy group of 3,4-ethylenedioxythiophene.

The reaction conditions such as reaction temperature and reaction time in a case of producing the thiophene compound represented by General Formula (3) can be appropriately determined depending on the kind of the thiophene compound represented by General Formula (3).

In the thiophene compound represented by General Formula (3), the imide salt (—SO₂—N⁻M—SO₂A) contained in the substituent (—X—SO₂—N⁻M—SO₂A) bonded to 3,4-ethylenedioxythiophene contains a cation (M). For this reason, the cation (M) can be easily exchanged for a desired cation by a method of dissolving the thiophene compound represented by General Formula (3) in a solvent and exchanging the kind of cation using a cation exchange resin corresponding to a desired cation.

As the solvent that is used in a case of exchanging the cation (M) contained in the thiophene compound represented by General Formula (3), it is possible to use, for example, 2-methylpyrrolidone, acetone, dimethylformamide, dimethylacetamide, tetrahydrofuran, and acetonitrile.

In addition, examples of the cation exchange resin that can be used in a case of exchanging the cation (M) contained in the thiophene compound represented by General Formula (3) include those described below.

For example, in a case where M in the thiophene compound represented by General Formula (3) is changed from —Na⁺ to —H⁺, it is possible to use LEWATIT MonoPlus S108H (manufactured by Lanxess AG), ORTHOLITE DS-01 (manufactured by ORGANO CORPORATION), AMBERLYST 15JS-HG DRY (manufactured by ORGANO CORPORATION), or the like as the cation exchange resin.

In addition, for example, in a case where M in the thiophene compound represented by General Formula (3) is changed from —H⁺ to —Na⁺, it is possible to use LEWATIT MonoPlus S108 (manufactured by Lanxess AG) or the like as the cation exchange resin.

In addition, since the imide salt contained in the thiophene compound represented by General Formula (3) contains a cation (M), the thiophene compound represented by General Formula (3) can be easily changed by a method of neutralizing the thiophene compound using an amine compound having a group corresponding to R¹ in the above formula —N⁺H(R¹)₃, such as ethanolamine as a neutralizing agent, for example, in a case where M in the thiophene compound represented by General Formula (3) is —H⁺ and it is desired to change M to an ammonium ion such as —N⁺H₃C₂H₄OH (—N⁺H(R¹)₃ (three R¹'s in the formula each independently represent a hydrogen atom or an alkyl group having 1 to 15 carbon atoms which may have a substituent)).

In addition, for example, in a case where M in the thiophene compound represented by General Formula (3) is —Na⁺ and it is desired to change M to an ammonium ion, M can be easily changed by a method of changing M of the thiophene compound represented by General Formula (3) from —Na⁺ to —H⁺ by the above-described method using a cation exchange resin and then carrying out neutralization using an amine compound corresponding to the desired ammonium ion.

### “Method of Producing Polymer”

The polymer according to the present embodiment can be produced by subjecting a raw material monomer containing a thiophene compound represented by General Formula (3) to chemical oxidative polymerization by using, for example, a polymerization initiator such as sodium persulfate or potassium persulfate and an oxidizing agent such as iron chloride by a publicly known method.

The polymerization conditions such as the reaction temperature, the reaction time, and the amount of polymerization initiator to be used in producing the polymer according to the present embodiment can be appropriately determined depending on the composition of the raw material monomer and the like.

The raw material monomer that is used in the method of producing the polymer according to the present embodiment may contain only one kind of thiophene compound represented by General Formula (3), or it may contain, as necessary, two or more kinds of thiophene compounds represented by General Formula (3). In addition, the raw material monomer may contain, as necessary, not only the thiophene compound represented by General Formula (3) but also one kind or two or more kinds of publicly known monomers in addition to the thiophene compound represented by General Formula (3).

Examples of the publicly known monomer that may be contained in the raw material monomer include 3,4-ethylenedioxythiophene, 2-(chloromethyl)-2,3-dihydrothieno[3,4-b]-1,4-dioxin, and 2,3-dihydrothieno[3,4-b][1,4] dioxin-2-yl)methanol.

The kind and content of the publicly known monomer can be appropriately determined depending on the use application of the target polymer, within a range in which the effect of the present invention can be obtained.

The polymer according to the present embodiment has the substituent (—X—SO₂—N⁻M—SO₂A) having a dopant function derived from the thiophene compound represented by General Formula (3). As a result, in the polymer according to the present embodiment, the imide salt (—SO₂—N⁻M—SO₂A) contained in the substituent having a dopant function contains a cation which is M in General Formula (2).

For this reason, in the polymer according to the present embodiment, the cation (M) can be easily exchanged for a desired cation similarly to the case of the thiophene compound represented by General Formula (3).

Specifically, it can be easily exchanged for a desired cation by a method of dissolving the polymer according to the present embodiment and exchanging the kind of cation using a cation exchange resin corresponding to the desired cation.

Examples of the solvent that can be used to dissolve the polymer according to the present embodiment include 2-methylpyrrolidone, acetone, dimethylformamide, dimethylacetamide, tetrahydrofuran, and acetonitrile.

In addition, for example, in a case where M in the polymer according to the present embodiment is —H⁺ and it is desired to change M to —N⁺H₃C₂H₄OH, the polymer according to the present embodiment can be easily changed by a method of carrying out neutralization using ethanolamine as a neutralizing agent.

### “Conductive Member”

The conductive member according to the present embodiment contains the polymer according to the present embodiment. Hereinafter, the conductive member according to the present embodiment will be described taking a sheet-shaped conductive member as an example. It is noted that the conductive member according to the present embodiment may have any shape as long as it contains the polymer according to the present embodiment, where the shape is not limited to a sheet shape.

The polymer according to the present embodiment which is contained in the conductive member according to the present embodiment may be only one kind or may be two or more kinds. In addition, the conductive member according to the present embodiment may contain, together with the polymer according to the present embodiment, one kind or two or more kinds of publicly known polymers other than the polymer according to the present embodiment, as necessary. In addition, the conductive member according to the present embodiment may contain, as necessary, one kind or two or more kinds of publicly known conductive members together with the polymer according to the present embodiment.

Examples of the publicly known polymer that may be contained in the conductive member according to the present embodiment include a publicly known conductive polymer such as a composite body of poly-3,4-ethylenedioxythiophene and polystyrenesulfonic acid (PEDOT/PSS), an acrylic resin such as polymethyl methacrylate, a vinyl acetal resin such as polyvinyl butyral, a thermosetting resin such as phenol novolac or an epoxy resin, a silicone resin such as polydimethylsiloxane, a fluororesin such as polyvinyl vinylidene fluoride, polystyrene, styrene butadiene rubber, polyacrylonitrile, polyethylene glycols, ethyl cellulose, and a cellulose nanofiber.

Examples of the publicly known conductive member that may be contained in the conductive member according to the present embodiment include metal materials consisting of copper, gold, nickel, and aluminum, and carbon materials such as graphite, activated carbon, graphene, and a carbon nanotube.

The conductive member according to the present embodiment can be produced, for example, by the method described below.

The conductive member containing the polymer according to the present embodiment which serves as the conductive member according to the present embodiment is dissolved in a publicly known solvent such as dimethylformamide, acetone, dimethylacetamide, tetrahydrofuran, or acetonitrile to prepare a coating liquid. Next, the coating liquid is applied onto a peelable base material to a predetermined thickness to form a coating film. It is possible to use, as the base material, a publicly known base material consisting of a resin film such as polyethylene terephthalate (PET). As the coating method for the coating liquid, a publicly known method can be used depending on the coating thickness, the viscosity of the coating liquid, or the like. Thereafter, the coating film is dried to remove the solvent in the coating film, and then the coating film is peeled off from the base material to obtain a sheet-shaped conductive member.

### “Electronic Part”

The electronic part according to the present embodiment includes the conductive member according to the present embodiment.

Specific examples of the electronic part according to the present embodiment include a sensor, a capacitor, an actuator, a lithium ion secondary battery, and a bioelectrode.

The electronic part according to the present embodiment can be produced by providing a conductive member containing the polymer according to the present embodiment by a publicly known method.

The polymer according to the present embodiment contains at least one structural unit selected from a structural unit represented by General Formula (1) and a structural unit represented by General Formula (2). For this reason, the polymer according to the present embodiment is a self-doped type conductive polymer having low hydrophilicity and good lipophilicity, and it can be suitably used as a material that is capable of forming a conductive member having sufficient conductivity and excellent water resistance. In addition, since the polymer according to the present embodiment is a self-doped type conductive polymer, it does not require a dopant from the outside and does not undergo dopant migration, and thus the polymer according to the present embodiment has excellent stability.

In addition, the thiophene compound according to the present embodiment is represented by General Formula (3). For this reason, in a case of polymerizing a raw material monomer containing the thiophene compound according to the present embodiment, it is possible to produce the polymer according to the present embodiment, which is a self-doped type conductive polymer that has sufficient conductivity, has low hydrophilicity, and has good lipophilicity.

In addition, in the polymer and thiophene compound according to the present embodiment, the imide salt (—SO₂—N⁻M—SO₂A) contained in the substituent (—X—SO₂—N⁻M—SO₂A) bonded to 3,4-ethylenedioxythiophene contains a cation (M). For this reason, in the polymer and thiophene compound according to the present embodiment, the cation (M) can be easily changed to a desired cation by using a method of carrying out dissolution in a solvent to exchange the kind of cation using a cation exchange resin corresponding to the desired cation or a method of carrying out neutralization using a neutralizing agent.

In addition, the conductive member according to the present embodiment, and the electronic part including the conductive member contain the polymer according to the present embodiment. For this reason, the conductive member according to the present embodiment and electronic part including the conductive member have excellent water resistance.

As described above, the embodiments of the present invention have been described in detail. However, each of the configurations and the combination thereof in each embodiment are examples, and thus additions, omissions, substitutions, and other modifications of the configuration can be made without departing from the spirit of the present invention.

### EXAMPLES

### Example 1

### “Method of Producing Thiophene Compound”

(Synthesis of Intermediate Compound (a-1))

Under a nitrogen stream, 5.53 g of trifluoromethanesulfonamide (37.1 mmol, manufactured by Tokyo Chemical Industry Co., Ltd.), 2.36 g of anhydrous sodium carbonate (22.2 mmol, manufactured by KISHIDA CHEMICAL Co., Ltd.), and 20 mL of dehydrated acetonitrile (manufactured by Kanto Chemical Co., Inc.) were added to a two-necked flask equipped with a reflux condenser, and the flask was cooled with ice water. Thereafter, a mixed solution of 5.00 g of 4-(bromomethyl)benzenesulfonyl chloride (18.6 mmol, manufactured by Tokyo Chemical Industry Co., Ltd.) represented by Formula (a-0) and 20 mL of dehydrated acetonitrile was dropwise added to the flask, and stirring was carried out while carrying out heating at 70° C. for 12 hours.

After cooling, 100 mL of ethyl acetate was added thereto, the inorganic salt was filtered out by filtration, and the filtrate was concentrated by a rotary evaporator to obtain a crude product. This was dissolved in acetone and purified by reprecipitation from cyclopentyl methyl ether. As a result, a white solid (yield: 5.32 g) consisting of an intermediate compound represented by Formula (a-1) was obtained.

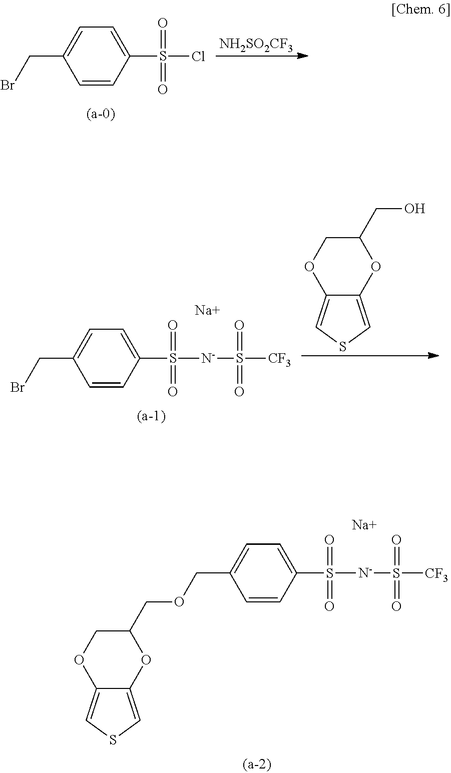

(Synthesis of Thiophene Compound (a-2))

Under a nitrogen stream, 0.46 g of 55% sodium hydride (10.5 mmol, manufactured by Kanto Chemical Co., Inc.), 1.72 g of (2,3-dihydrothieno[3,4-b][1,4]dioxin-2-yl)methanol (9.98 mmol, manufactured by Kanto Chemical Co., Inc.), and 20 mL of dehydrated tetrahydrofuran (manufactured by Kanto Chemical Co., Inc.) were added to a two-necked flask equipped with a reflux condenser, and reflux was carried out for 1 hour.

After cooling, a mixed solution of 3.63 g (8.99 mmol) of the intermediate compound (a-1) synthesized by the above reaction and 10 mL of dehydrated tetrahydrofuran was dropwise added to the flask, and heating under reflux was carried out for 16 hours.

After cooling, 200 mL of ethyl acetate was added thereto, the inorganic salt was filtered out by filtration, and the filtrate was concentrated by a rotary evaporator to obtain a crude product. This was dissolved in acetone and purified by reprecipitation from cyclopentyl methyl ether and silica column chromatography. As a result, a white solid (yield: 2.85 g) consisting of a thiophene compound represented by Formula (a-2) was obtained.

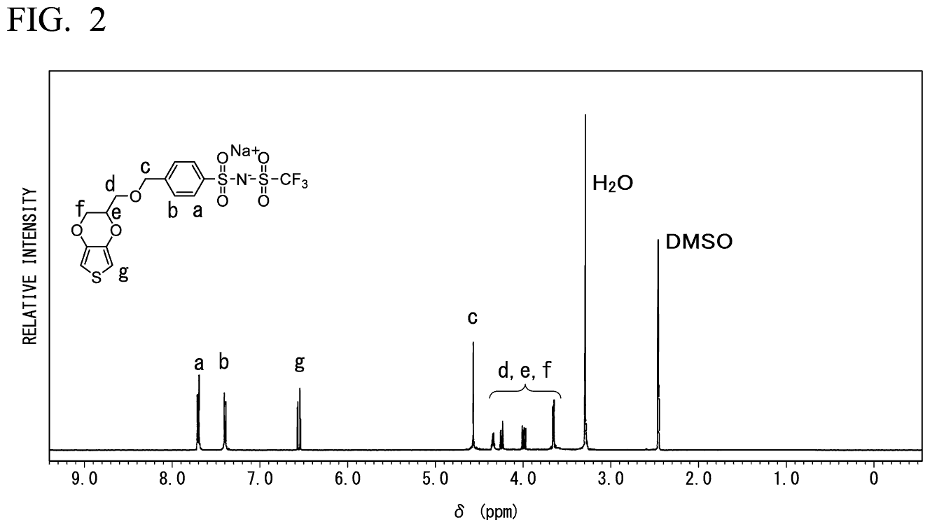

The obtained thiophene compound was subjected to ¹H-NMR (nuclear magnetic resonance) measurement. For the ¹H-NMR measurement, JNM-ECA500 (manufactured by JEOL RESONANCE Inc.) was used as an NMR apparatus. In addition, for the ¹H-NMR measurement, dimethyl sulfoxide d6 (DMSO-d6), which is a deuterated solvent, was used as a solvent. The results are shown in FIG. 2 and below. FIG. 2 is a ¹H-NMR measurement chart of a thiophene compound synthesized in Example 1.

In addition, the obtained thiophene compound was subjected to mass spectrometry. Mass spectrometry was carried out by an electrospray ionization (ESI) method using Agilent 6100 Quadrupole MS (manufactured by Agilent Technologies, Inc.) as a mass spectrometer.

From the results of the 1H-NMR measurement and the mass spectrometry shown below, it could be confirmed that the synthesized thiophene compound is the thiophene compound represented by Formula (a-2).

¹H-NMR (500 MHz, heavy DMSO) δ (ppm) 7.71-7.68 (2H, m), 7.40-7.39 (2H, m), 6.57-6.54 (2H, m), 4.57 (2H, s), 4.36-4.33 (1H, m), 4.26-4.23 (1H, m), 4.02-3.98 (1H, m), 3.66-3.65 (2H, m)

m/z 471 ([M—] C₁₅H₁₃F₃NO₇S)

### (Synthesis of Polymer (P-1))

97.7 mg (0.197 mmol) of the thiophene compound represented by Formula (a-2) was added to a glass bottle as a raw material monomer and dissolved in 900 mg of pure water. Next, a mixed aqueous solution of 19.2 mg of FeCl₃ (0.118 mmol, manufactured by FUJIFILM Wako Pure Chemical Corporation), 93.9 mg of sodium persulfate (0.395 mmol, Junsei Chemical Co., Ltd.), and 900 mg of pure water were added thereto, and the glass bottle was capped, and the mixture was vigorously stirred at room temperature for 3 hours to cause chemical oxidative polymerization. This produced a dark blue solid.

Thereafter, 2 mL of pure water was added to the glass bottle, and the dark blue solid was recovered by filtration. The dark blue solid was washed with pure water to remove impurities such as inorganic salts. The washed dark blue solid was added to acetone and subjected to an ultrasonic treatment, and inorganic salts were filtered out using a filter having a pore diameter of 0.22 μm. The filtrate was concentrated by a rotary evaporator to obtain a target polymer represented by Formula (P-1).

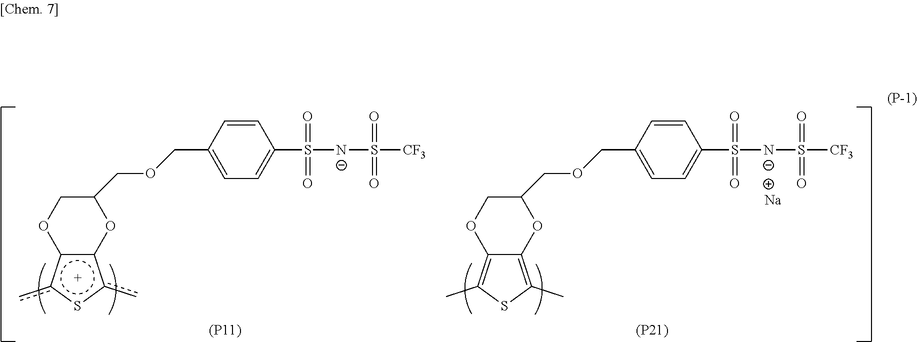

It was confirmed that the polymer of Example 1 is in a doped state by the imide salt that serves as a dopant by an attenuated total reflection method (ATR method) using a Fourier transform infrared spectrophotometer (FT-IR) (Nicolet iS10, manufactured by Thermo Fisher Scientific, Inc.), and the molecular structure of the polymer of Example 1 was identified.

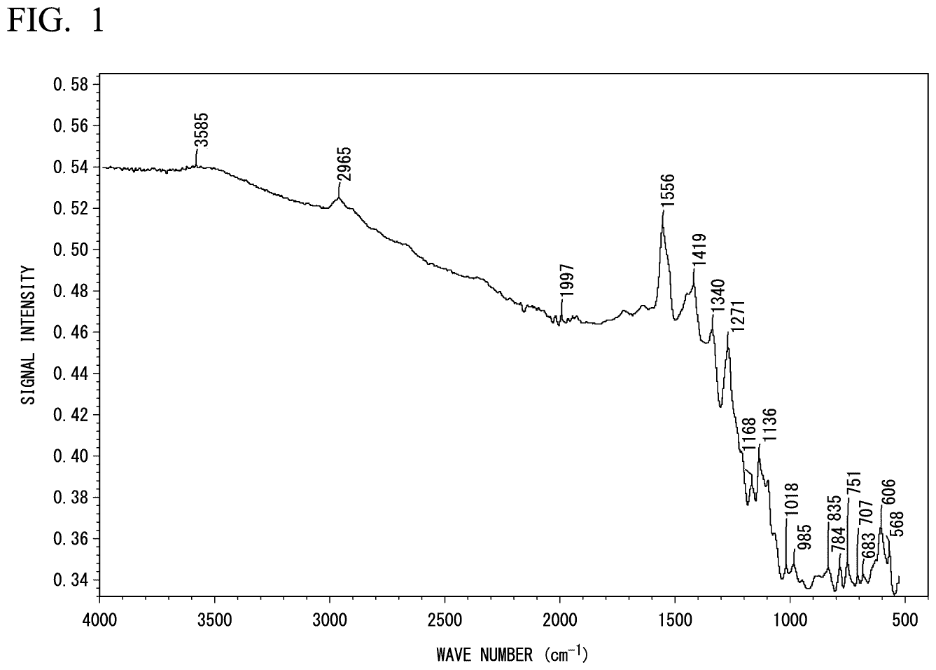

FIG. 1 is a graph showing the results obtained by analyzing a polymer of Example 1 by an attenuated total reflection method using a Fourier transform infrared spectrophotometer (FT-IR). As shown in FIG. 1, a characteristic band absorption due to doping was observed in a wave number range of 4,000 to 1,800 cm⁻¹.

As a result, it was confirmed that the polymer of Example 1 is a polymer represented by Formula (P-1) which contains a structural unit represented by Formula (P11) and a structural unit represented by Formula (P21).

In addition, the number average molecular weight (Mn), the weight average molecular weight (Mw), and the dispersity (Mw/Mn) of the polymer of Example 1 were determined by the methods described below.

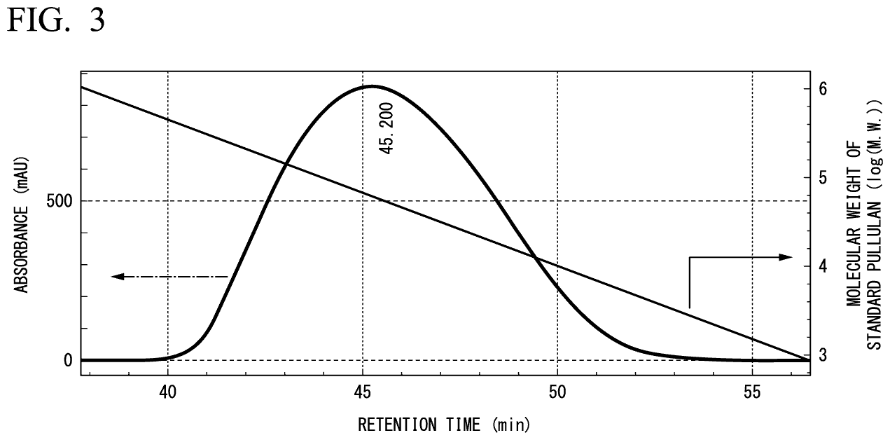

First, the polymer of Example 1 was dissolved in the following mobile phase so that the concentration thereof was 0.5 W/V %, and then filtered using a polytetrafluoroethylene (PTFE) filter having a sieve opening of 0.45 μm to obtain a sample solution. The obtained sample solution was analyzed using gel permeation chromatography (GPC) (LC-2050C; manufactured by Shimadzu Corporation) under the measurement conditions shown below. The results are shown in FIG. 3.

### (Measurement Condition)

Injection volume: 20 μL
Column: TSKgel α-M (manufactured by TOSOH Corporation) two columns+guard column α
Column temperature: 50° C.
Mobile phase: DMSO (dimethyl sulfoxide) solution containing 50 mM tetrabutylammonium bromide
Flow rate: 0.3 ml/min
Detector: UV detector 620 nm (photodiode array detector)
Standard substance: Pullulan (Showa Denko K.K.)

FIG. 3 is a graph showing is a graph showing the results obtained by analyzing the polymer of Example 1 by gel permeation chromatography (GPC), together with the relationship between the weight average molecular weight (Mw) of pullulan, which is a standard substance, and the retention time (min).

The polymer of Example 1 was analyzed using GPC, and the obtained results were such that the number average molecular weight (Mn) calculated was 33,300, the weight average molecular weight (Mw) calculated was 74,500, and the dispersity (Mw/Mn) calculated was 2.23.

### Example 2

The polymer represented by Formula (P-1) which had been produced in the same manner as in Example 1 was dissolved in 2-methylpyrrolidone, and the kind of cation was exchanged from —Na⁺ to —H⁺ using a cation exchange resin consisting of LEWATIT MonoPlus S108H (manufactured by Lanxess AG), whereby a target polymer represented by Formula (P-2) was obtained.

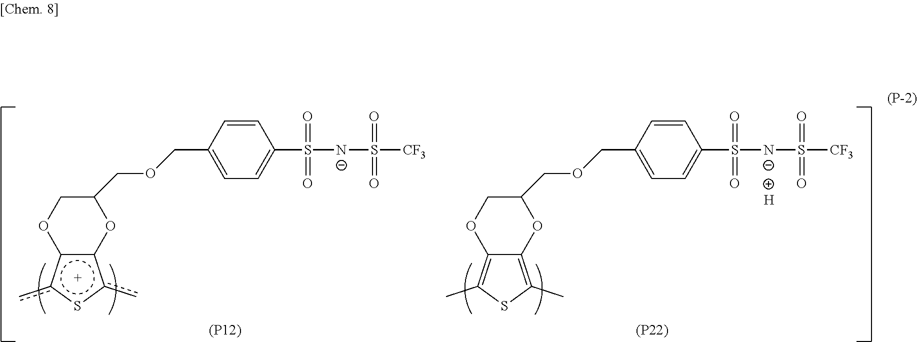

In the same manner as in the case of the polymer of Example 1, it was confirmed that the polymer of Example 2 is in a doped state by the imide salt that serves as a dopant by an attenuated total reflection method (ATR method) using the Fourier transform infrared spectrophotometer (FT-IR), and the molecular structure of the polymer of Example 2 was identified.

As a result, it was confirmed that the polymer of Example 2 is a polymer represented by Formula (P-2) which contains a structural unit represented by Formula (P12) and a structural unit represented by Formula (P22).

### Example 3

The polymer represented by Formula (P-2) which had been produced in the same manner as in Example 2 was neutralized with ethanolamine to change the kind of cation from —H⁺ to —N⁺H₃C₂H₄OH, whereby a target polymer represented by Formula (P-3) was obtained.

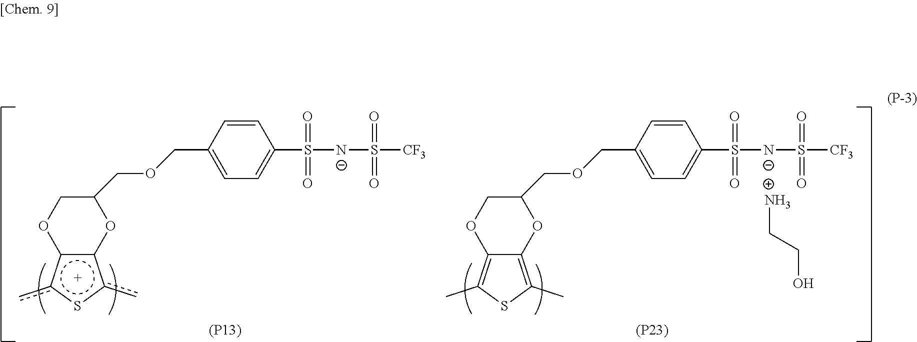

In the same manner as in the case of the polymer of Example 1, it was confirmed that the polymer of Example 3 is in a doped state by the imide salt that serves as a dopant by an attenuated total reflection method (ATR method) using the Fourier transform infrared spectrophotometer (FT-IR), and the molecular structure of the polymer of Example 3 was identified.

As a result, it was confirmed that the polymer of Example 3 is a polymer represented by Formula (P-3) which contains a structural unit represented by Formula (P13) and a structural unit represented by Formula (P23).

### Example 4

A target polymer represented by Formula (P-4) was obtained in the same manner as in the case of the polymer represented by Formula (P-1) which had been produced in Example 1, except that pentafluoroethanesulfonamide (manufactured by Tokyo Chemical Industry Co., Ltd.) was used instead of trifluoromethanesulfonamide in a case where the intermediate compound (a-1) was synthesized.

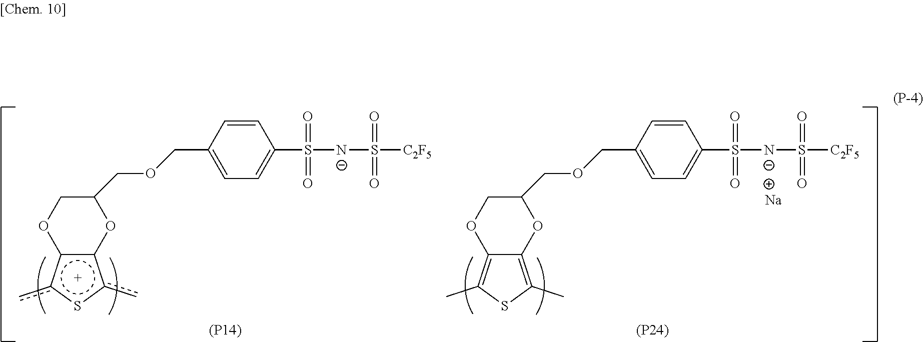

In the same manner as in the case of the polymer of Example 1, it was confirmed that the polymer of Example 4 is in a doped state by the imide salt that serves as a dopant by an attenuated total reflection method (ATR method) using the Fourier transform infrared spectrophotometer (FT-IR), and the molecular structure of the polymer of Example 4 was identified.

As a result, it was confirmed that the polymer of Example 4 is a polymer represented by Formula (P-4) which contains a structural unit represented by Formula (P14) and a structural unit represented by Formula (P24).

### Example 5

The polymer represented by Formula (P-4) which had been produced in the same manner as in Example 4 was dissolved in 2-methylpyrrolidone, and the kind of cation was exchanged from —Na⁺ to —H⁺ using a cation exchange resin consisting of LEWATIT MonoPlus S108H, whereby a target polymer represented by Formula (P-5) was obtained.

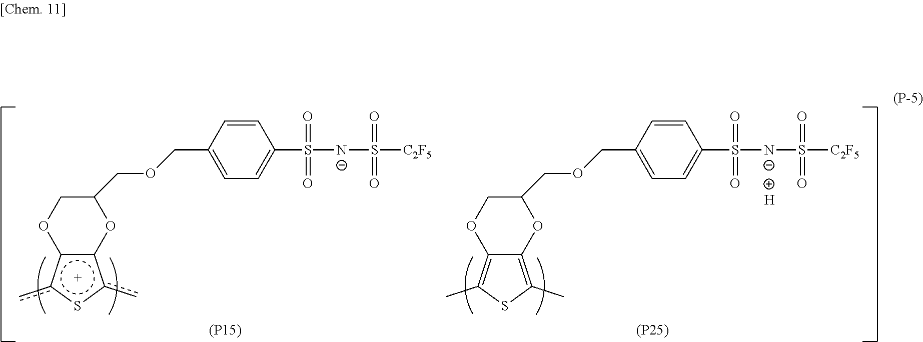

In the same manner as in the case of the polymer of Example 1, it was confirmed that the polymer of Example 5 is in a doped state by the imide salt that serves as a dopant by an attenuated total reflection method (ATR method) using the Fourier transform infrared spectrophotometer (FT-IR), and the molecular structure of the polymer of Example 5 was identified.

As a result, it was confirmed that the polymer of Example 5 is a polymer represented by Formula (P-5) which contains a structural unit represented by Formula (P15) and a structural unit represented by Formula (P25).

### Example 6

A target polymer represented by Formula (P-6) was obtained in the same manner as in the case of the polymer represented by Formula (P-1) which had been produced in Example 1, except that 4-fluorobenzenesulfonyl chloride (manufactured by Tokyo Chemical Industry Co., Ltd.) was used instead of 4-(bromomethyl)benzenesulfonyl chloride represented by Formula (a-0) in a case where the intermediate compound (a-1) was synthesized.

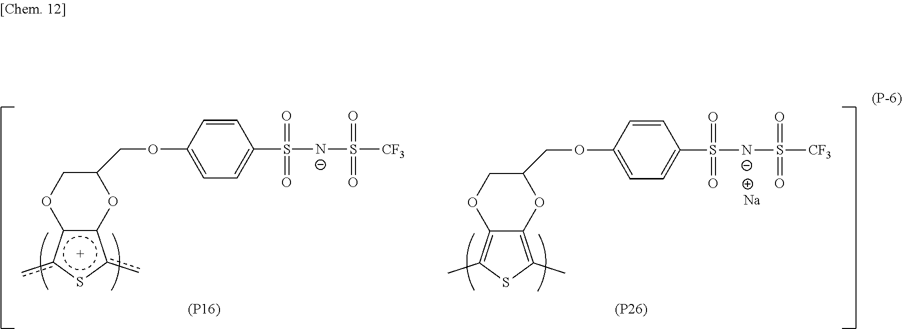

In the same manner as in the case of the polymer of Example 1, it was confirmed that the polymer of Example 6 is in a doped state by the imide salt that serves as a dopant by an attenuated total reflection method (ATR method) using the Fourier transform infrared spectrophotometer (FT-IR), and the molecular structure of the polymer of Example 6 was identified.

As a result, it was confirmed that the polymer of Example 6 is a polymer represented by Formula (P-6) which contains a structural unit represented by Formula (P16) and a structural unit represented by Formula (P26).

### Example 7

The polymer represented by Formula (P-6) which had been produced in the same manner as in Example 6 was dissolved in 2-methylpyrrolidone, and the kind of cation was exchanged from —Na⁺ to —H⁺ using a cation exchange resin consisting of LEWATIT MonoPlus S108H, whereby a target polymer represented by Formula (P-7) was obtained.

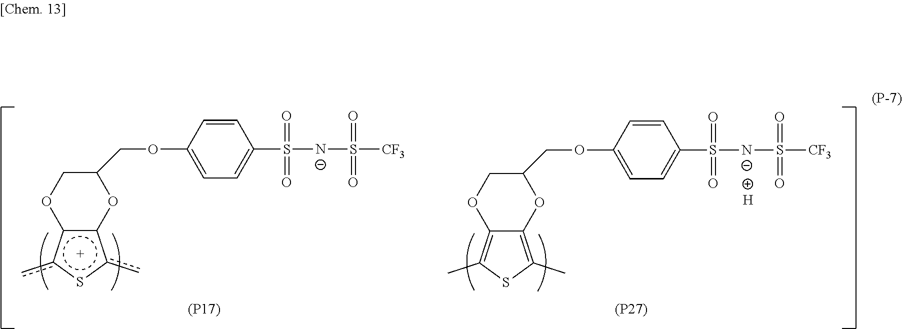

In the same manner as in the case of the polymer of Example 1, it was confirmed that the polymer of Example 7 is in a doped state by the imide salt that serves as a dopant by an attenuated total reflection method (ATR method) using the Fourier transform infrared spectrophotometer (FT-IR), and the molecular structure of the polymer of Example 7 was identified.

As a result, it was confirmed that the polymer of Example 7 is a polymer represented by Formula (P-7) which contains a structural unit represented by Formula (P17) and a structural unit represented by Formula (P27).

### Example 8

A target polymer represented by Formula (P-8) was obtained in the same manner as in the case of the polymer represented by Formula (P-1) which had been produced in Example 1, except that in a case where the intermediate compound (a-1) was synthesized, pentafluoroethanesulfonamide (manufactured by Tokyo Chemical Industry Co., Ltd.) was used instead of trifluoromethanesulfonamide and 4-fluorobenzenesulfonyl chloride (manufactured by Tokyo Chemical Industry Co., Ltd.) was used instead of 4-(bromomethyl)benzenesulfonyl chloride represented by Formula (a-0).

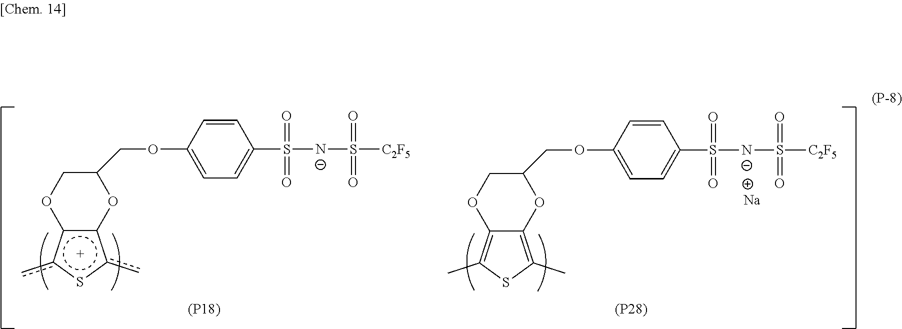

In the same manner as in the case of the polymer of Example 1, it was confirmed that the polymer of Example 8 is in a doped state by the imide salt that serves as a dopant by an attenuated total reflection method (ATR method) using the Fourier transform infrared spectrophotometer (FT-IR), and the molecular structure of the polymer of Example 8 was identified.

As a result, it was confirmed that the polymer of Example 8 is a polymer represented by Formula (P-8) which contains a structural unit represented by Formula (P18) and a structural unit represented by Formula (P28).

### Example 9

The polymer represented by Formula (P-8) which had been produced in the same manner as in Example 8 was dissolved in 2-methylpyrrolidone, and the kind of cation was exchanged from —Na⁺ to —H⁺ using a cation exchange resin consisting of LEWATIT MonoPlus S108H, whereby a target polymer represented by Formula (P-9) was obtained.

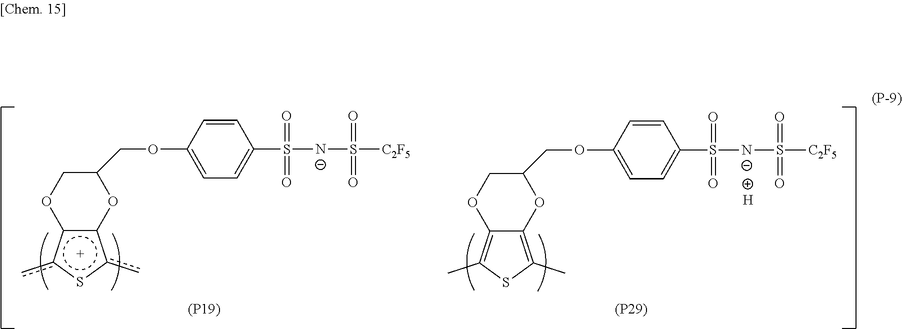

In the same manner as in the case of the polymer of Example 1, it was confirmed that the polymer of Example 9 is in a doped state by the imide salt that serves as a dopant by an attenuated total reflection method (ATR method) using the Fourier transform infrared spectrophotometer (FT-IR), and the molecular structure of the polymer of Example 9 was identified.

As a result, it was confirmed that the polymer of Example 9 is a polymer represented by Formula (P-9) which contains a structural unit represented by Formula (P19) and a structural unit represented by Formula (P29).

### Example 10

A target polymer represented by Formula (P-10) was obtained in the same manner as in the case of the polymer represented by Formula (P-1) which had been produced in Example 1, except that methanesulfonamide (manufactured by Tokyo Chemical Industry Co., Ltd.) was used instead of trifluoromethanesulfonamide in a case where the intermediate compound (a-1) was synthesized.

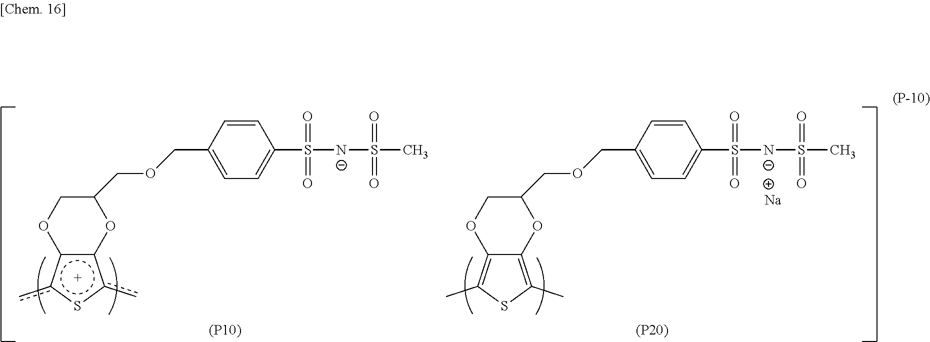

In the same manner as in the case of the polymer of Example 1, it was confirmed that the polymer of Example 10 is in a doped state by the imide salt that serves as a dopant by an attenuated total reflection method (ATR method) using the Fourier transform infrared spectrophotometer (FT-IR), and the molecular structure of the polymer of Example 10 was identified.

As a result, it was confirmed that the polymer of Example 10 is a polymer represented by Formula (P-10) which contains a structural unit represented by Formula (P10) and a structural unit represented by Formula (P20).

### Example 11

A target polymer represented by Formula (P-11) was obtained in the same manner as in the case of the polymer represented by Formula (P-1) which had been produced in Example 1, except that a mixture containing, as raw material monomers, the thiophene compound represented by Formula (a-2) and 3,4-ethylenedioxythiophene at a molar ratio of 95:5 (thiophene compound represented by Formula (a-2): 3,4-ethylenedioxythiophene) was used instead of the thiophene compound represented by Formula (a-2) in a case where the polymer (P-1) was synthesized.

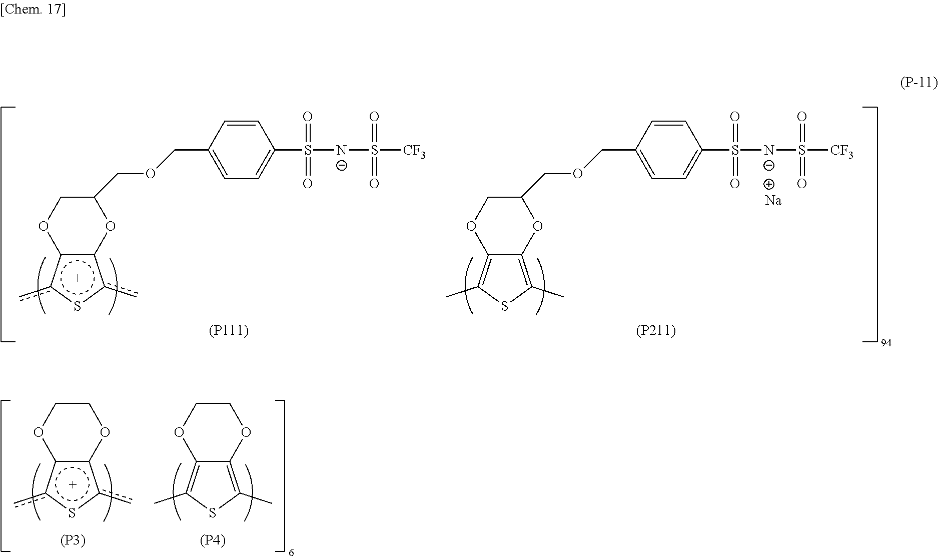

In the same manner as in the case of the polymer of Example 1, it was confirmed that the polymer of Example 11 is in a doped state by the imide salt that serves as a dopant by an attenuated total reflection method (ATR method) using the Fourier transform infrared spectrophotometer (FT-IR), and the molecular structure of the polymer of Example 11 was identified.

As a result, it was confirmed that the polymer of Example 11 is a polymer represented by Formula (P-11) which contains a structural unit represented by Formula (P111), a structural unit represented by Formula (P211), a structural unit represented by Formula (P3), and a structural unit represented by Formula (P4).

In addition, it was confirmed that the ratio of the total of the structural units represented by Formula (P111) and (P211) to the total of the structural units represented by Formula (P3) and Formula (P4) in the polymer represented by Formula (P-11) is 94:6 (Formula (P111) and Formula (P211):Formula (P3) and Formula (P4)) in terms of molar ratio from the results of the ¹H-NMR measurement.

### Example 12

A target polymer represented by Formula (P-12) was obtained in the same manner as in the case of the polymer represented by Formula (P-1) which had been produced in Example 1, except that 3-chloropropanesulfonyl chloride (manufactured by Sigma-Aldrich Co., LLC) was used instead of 4-(bromomethyl)benzenesulfonyl chloride in a case where the intermediate compound (a-1) was synthesized.

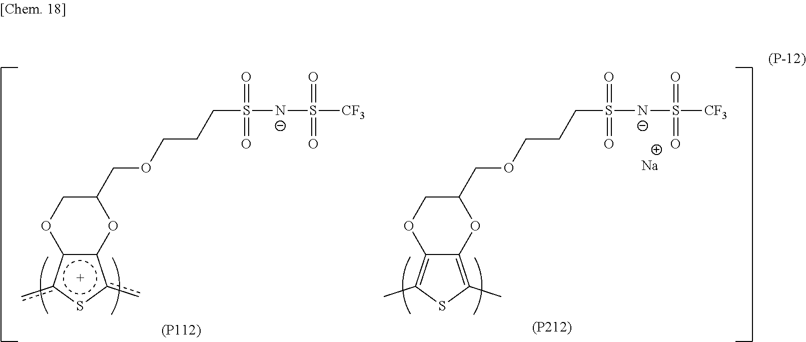

In the same manner as in the case of the polymer of Example 1, it was confirmed that the polymer of Example 12 is in a doped state by the imide salt that serves as a dopant by an attenuated total reflection method (ATR method) using the Fourier transform infrared spectrophotometer (FT-IR), and the molecular structure of the polymer of Example 12 was identified.

As a result, it was confirmed that the polymer of Example 12 is a polymer represented by Formula (P-12) which contains a structural unit represented by Formula (P112) and a structural unit represented by Formula (P212).

### Comparative Example 1

Using the method described in Patent Document 1, a polymer represented by Formula (Q-1) which contains a structural unit represented by Formula (Q11) and a structural unit represented by Formula (Q21) was obtained.

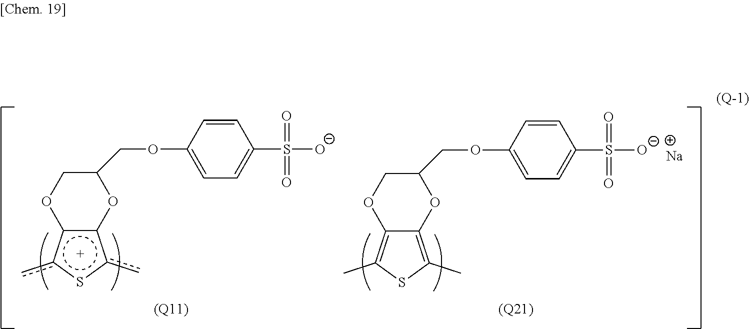

### Comparative Example 2

Using the method described in Patent Document 2, a polymer represented by Formula (Q-2) which contains a structural unit represented by Formula (Q12) and a structural unit represented by Formula (Q22) was obtained.

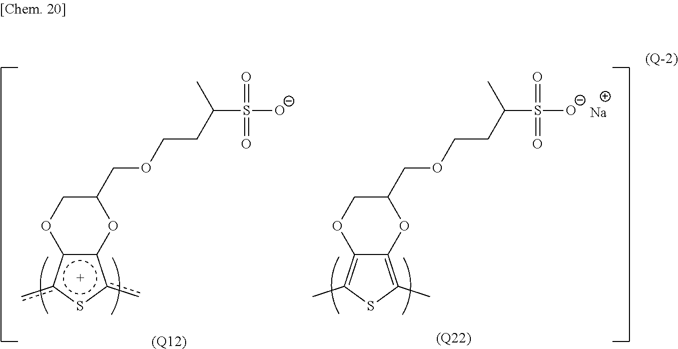

For the polymers (P-1) to (P-12), (Q-1), and (Q-2) in Example 1 to Example 12 and Comparative Example 1 and Comparative Example 2, Table 1 shows L and Ar (X in a case of being applied to General Formula (2) for (P-12)) in a case of being applied to General Formula (L—Ar), and M and A in a case of being applied to General Formula (2), respectively.

In addition, each of the polymers (P-1) to (P-12), (Q-1), and (Q-2) in Example 1 to Example 12 and Comparative Example 1 and Comparative Example 2 was used to form a conductive film (conductive member) by the method shown below, and the water resistance was evaluated.

### “Evaluation of Water Resistance”

The polymer was dissolved in dimethylformamide to prepare a coating liquid. Next, the coating liquid was applied onto a PET film and dried at 100° C. to obtain a conductive film (conductive member) having a thickness of 5 μm.

The conductance of the obtained conductive film on the PET film was measured by using a four-terminal method, and the measured conductance was defined as the initial conductance. The results are shown in Table 1.

Thereafter, the PET film on which the conductive film was formed was immersed in pure water for 30 minutes, taken out of the water, and subjected to air drying to obtain a sample after immersion in water.

The appearance of the sample after immersion in water was visually observed to evaluate the presence or absence of the peeling-off of the conductive film from the PET film. The results are shown in Table 1.

In addition, the conductance of the conductive film on the PET film in the sample after immersion in water was measured using a four-terminal method, and this measured conductance was defined as the conductance after immersion in water. Then, using the initial conductance and the conductance after immersion in water, the change in conductance after immersion in water ((conductance after immersion in water/initial conductance)×100 (%)) was calculated and evaluated based on the criteria shown below. The results are shown in Table 1.

### [Criteria]

Change in conductance is ±20% or less: A
Change in conductance is ±50% or less: B
Change in conductance is ±50% or more: C
Unmeasurable (the conductive film has been peeled off from the PET film due to immersion in water): D

TABLE 1

| | | | | | | | Change in | Appearance of |
|---|---|---|---|---|---|---|---|---|
| | | | | | | Initial | conductance | conductive film |
| | | | | | | conductance | after immersion | after immersion |
| | Polymer | L | Ar | M | A | (S/cm) | in water | in water |
| Example 1 | P-1 | CH₂OCH₂ | Phenylene group | Na⁺ | CF₃ | 22.7 | A | No change |
| Example 2 | P-2 | CH₂OCH₂ | Phenylene group | H⁺ | CF₃ | 30.7 | A | No change |
| Example 3 | P-3 | CH₂OCH₂ | Phenylene group | N⁺H₃C₂H₄OH | CF₃ | 22.0 | A | No change |
| Example 4 | P-4 | CH₂OCH₂ | Phenylene group | Na⁺ | C₂F₅ | 16.7 | A | No change |
| Example 5 | P-5 | CH₂OCH₂ | Phenylene group | H⁺ | C₂F₅ | 21.7 | A | No change |
| Example 6 | P-6 | CH₂O | Phenylene group | Na⁺ | CF₃ | 21.7 | A | No change |
| Example 7 | P-7 | CH₂O | Phenylene group | H⁺ | CF₃ | 23.2 | A | No change |
| Example 8 | P-8 | CH₂O | Phenylene group | Na⁺ | C₂F₅ | 16.7 | A | No change |
| Example 9 | P-9 | CH₂O | Phenylene group | H⁺ | C₂F₅ | 14.7 | A | No change |
| Example 10 | P-10 | CH₂OCH₂ | Phenylene group | Na⁺ | CH₃ | 15.8 | B | No change |
| Example 11 | P-11 | CH₂OCH₂ | Phenylene group | Na⁺ | CF₃ | 42.7 | A | No change |
| Example 12 | P-12 | CH₂OCH₂CH₂CH₂ | — | Na⁺ | CF₃ | 19.1 | B | No change |
| Comparative | Q-1 | CH₂O | Phenylene group | Na⁺ | — | 8.2 | D | No conductive |
| Example 1 | | | | | | | | film on PET |
| Comparative | Q-2 | — | — | Na⁺ | — | 130.0 | D | No conductive |
| Example 2 | | | | | | | | film on PET |

As shown in Table 1, in all of the conductive films containing the polymers (P-1) to (P-12) of Examples 1 to 12, sufficient initial conductance was obtained, the change in conductance after immersion in water was evaluated as A or B, and there was no peeling-off of the conductive film from the PET film in the samples after immersion in water.

In addition, in the conductive film containing the polymer (P-1) in which A is a perfluoroalkyl group (CF₃), the change in conductance after immersion in water was evaluated as A, and the water resistance was good as compared with a conductive film containing the polymer (P-10) in which A is an alkyl group (CH₃) that does not have a substituent.

In addition, in the conductive film containing the polymer (P-1) in which Ar is a phenylene group, the change in conductance after immersion in water was evaluated as A, and the water resistance was good as compared with a conductive film containing a polymer (P-12) in which X in a case of being applied to General Formula (2) has a chain structure (CH₂OCH₂CH₂CH₂) obtained by subjecting two alkylene groups to ether bonding (in other words, the phenylene group in the polymer (P-1) is replaced with —CH₂CH₂—).

On the other hand, the conductive film containing the polymer (Q-1) of Comparative Example 1 had insufficient initial conductance, the conductive film was peeled off from the PET film in the sample after immersion in water, and the water resistance was insufficient.

In addition, although the conductive film containing the polymer (Q-2) of Comparative Example 2 had a high initial conductance, the conductive film was peeled off from the PET film in the sample after immersion in water, and the water resistance was insufficient.

1. A polymer comprising:
at least one structural unit selected from a structural unit represented by General Formula (1) and a structural unit represented by General Formula (2), 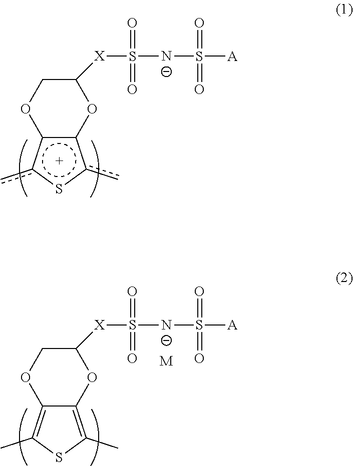
(in General Formula (1), X represents a divalent linking group, and A represents a halogen atom or an alkyl group having 1 to 5 carbon atoms which may have a substituent)
(in General Formula (2), X represents a divalent linking group, A represents a halogen atom or an alkyl group having 1 to 5 carbon atoms which may have a substituent, and M represents a cation).

2. The polymer according to claim 1,
wherein in the structural unit represented by General Formula (1) and the structural unit represented by General Formula (2), X is a divalent linking group represented by General Formula (L—Ar),
(in General Formula (L—Ar), L represents a single bond or a divalent linking group, and Ar represents any one selected from a phenylene group which may have a substituent, a biphenylene group which may have a substituent, or a naphthylene group which may have a substituent).

3. The polymer according to claim 2,
wherein L is represented by General Formula (L-a),
Ar is a phenylene group, and
A is a fluorine atom or an alkyl group having 1 to 5 carbon atoms, in which at least a part of hydrogen atoms are substituted with a fluorine atom, 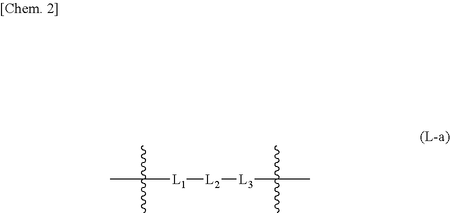
(in General Formula (L-a), L₁, L₂, and L₃ each independently represent any one selected from a single bond, an alkylene group having 1 to 2 carbon atoms, or an ether bond).

4. The polymer according to claim 1,
wherein in the structural unit represented by General Formula (1) and the structural unit represented by General Formula (2), X has a chain structure which is obtained by subjecting a plurality of alkylene groups to ether bonding.

5. A thiophene compound represented by General Formula (3), 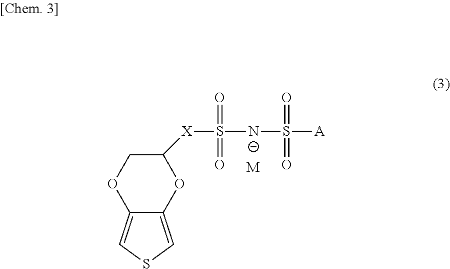
(in General Formula (3), X represents a divalent linking group, A represents a halogen atom or an alkyl group having 1 to 5 carbon atoms which may have a substituent, and M represents a cation.)

6. The thiophene compound according to claim 5,
wherein, in General Formula (3), X is a divalent linking group represented by General Formula (L—Ar),
(in General Formula (L—Ar), L represents a single bond or a divalent linking group, and Ar represents any one selected from a phenylene group which may have a substituent, a biphenylene group which may have a substituent, or a naphthylene group which may have a substituent).

7. The thiophene compound according to claim 6,
wherein, L is represented by General Formula (L-a),
Ar is a phenylene group, and
A is a fluorine atom or an alkyl group having 1 to 5 carbon atoms, in which at least a part of hydrogen atoms are substituted with a fluorine atom, 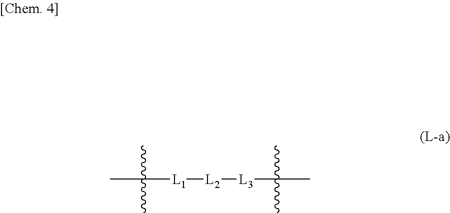
(in General Formula (L-a), L₁, L₂, and L₃ each independently represent any one selected from a single bond, an alkylene group having 1 to 2 carbon atoms, or an ether bond).

8. The thiophene compound according to claim 5,
wherein in General Formula (3), X has a chain structure which is obtained by subjecting a plurality of alkylene groups to ether bonding.

9. A conductive member comprising:
the polymer according to claim 1.

10. An electronic part comprising:
the conductive member according to claim 9.
